(19) United States
(12) Patent Application Publication
(10) Pub. No.: US 2008/0268481 A1
(43) Pub. Date: Oct. 30, 2008

(54) Sensitive Magnetic Catch Assay By Building a Strong Binding Couple

(75) Inventors: Menno Willem Jose Prins, Eindhoven (NL); Wendy Uyen Dittmer, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 12/094,791

(22) PCT Filed: Nov. 21, 2006
(86) PCT No.: PCT/IB06/54348

§ 371 Date: May 23, 2008

(30) Foreign Application Priority Data
Nov. 25, 2005 (EP) 05111287.8

(51) Int. Cl. G01N 33/53; B01J 19/00; G01N 33/553

(52) U.S. Cl. 435/7.92; 422/68.1; 436/526

(57) ABSTRACT

The invention relates to a sensor device for detecting a target in a sample suspected of comprising the target, which device is suitable for use in an assay such as a sandwich assay. The invention further relates to a method for detecting a target in a sample. The device comprises a sensor surface, which is functionalised with one moiety of a strong binding couple, which moiety preferably shows little or no cross reactivity with the target molecule.

### FIELD OF THE INVENTION

The invention relates to a device for detecting a target in a sample suspected of comprising the target. The invention further relates to a method for detecting a target in a sample.

### BACKGROUND TO THE INVENTION

A part of health care research involves developing diagnostic measurements to determine the presence or absence of specific proteins and other biological compounds such as DNA, RNA, hormones, metabolites, drugs etc as well as to determine the activity and function of active and catalytic biomolecules such as proteins, peptides, prions, enzymes, aptamers, ribozymes, and deoxyribozymes. Immunoassays are already used to determine the amount of specific proteins in body fluids to aid further diagnosis and treatment.

One of the well-known immunoassays is the two antibody “sandwich” ELISA. This assay is used to determine an antigen concentration in unknown samples. The sandwich ELISA requires two antibodies that bind to separate epitopes that do not overlap on the antigen. This can be accomplished with either two monoclonal antibodies that recognize discrete sites or affinity-purified polyclonal antibodies that have been raised to different epitopes on the antigen.

To utilize this assay, one antibody (the capture antibody) is purified and bound to a solid phase typically attached to the bottom of a well-plate. Antigen is then added and allowed to complex with the bound antibody. Unbound products are then removed with a wash, and a second antibody (the detection antibody), labelled with an enzyme is allowed to bind to the antigen, thus completing the “sandwich”. The assay is then made quantitative by measuring the amount of calorimetric substrate converted by the enzyme on second antibody bound to the matrix. Other labelling techniques including the use of a fluorescence or chemiluminescence labels are also commonly employed.

A sandwich format is sketched in FIG. 1. In this example the target 3 binds to the sensor surface 1 via binding moiety 2. The rate of binding dN/dt of targets to the sensor surface is approximately given by (unit s⁻¹):

$$\frac{d N}{d t} = {Ak}_{on} [ Cap ] [ T ] , \qquad ( 1 )$$

with A the area of the sensor surface (unit m²), kₒₙ the association constant of the binding process (unit m³/s), [Cap] the concentration of capture sites on the sensor surface (unit m⁻²), and [T] the concentration of targets in the solution directly above the sensor surface (unit m⁻³). In case of a very low target concentration, the assay of FIG. 1 may show the following drawbacks.

Firstly the sensor surface A is limited due to manufacturing costs, particularly when the sensor surface is a silicon chip.

Secondly actuation (e.g. mixing, shear flow) of the fluid is needed to avoid target-depletion above the sensor surface. This complicates the design of a device that is suitable for use in the method. The first capture process can be accelerated by increasing the capture area A, e.g. by capturing the target molecules onto the surface of particles suspended in solution. This is called a catch assay. An example is sketched in FIG. 2. Due to the small diameter of the particles, the total surface area can be very large and therefore the binding rate can be very high. However, a disadvantage of this assay is that a final detection step requires binding of the nanoparticle-with-target complex onto the sensor surface 1. This process can be very slow and inefficient due to steric hindrance, i.e. due to the fact that two large surfaces (the surface of particle 4 and sensor surface 1) need to be coupled via much smaller biological molecules, namely via target 3 and moieties 2 and 5.

Another standard assay format is the competitive assay. This assay is suitable for small target molecules which contain only one epitope and thus cannot be detected with a sandwich assay. In the competitive assay, target molecules compete with target homologues for binding sites typically either on a label or on a sensor surface. The binding sites occupied by the target homologue are then detected and this increases with decreasing target concentration. An example is depicted in FIG. 7 in which target molecule 3 competes with target homologue 9 for binding to moiety 5. At high concentrations of target molecule 3, most of the binding sites on moieties 4 become occupied by target 3. However at low concentrations of target 3, as shown in the second row of FIG. 7, most of the binding sites on moieties 4 become occupied by target homologue 9. Unlike the sandwich assay, the competitive assay measures the concentration of the target indirectly. For the example shown in FIG. 7, equation (2) is valid for the capture process of the target homologue:

$$\frac{d N}{d t} = {Ak}_{on} [ Cap ] [ TH ] , \qquad ( 2 )$$

where [TH] is the target homologue (unit m⁻²), with A the area of the sensor surface (unit m²), kₒₙ the association constant of the binding process (unit m³/s), and [Cap] the concentration of capture sites in solution (unit m⁻³⁾.

Similarly to the sandwich assay, the capture process of the target homologue can be accelerated by putting the target homologue on particles dispersed in solution (FIG. 8 showing in the top row a high target concentration and in the bottom row a low target concentration). However, the same difficulties as mentioned for the sandwich assay are encountered, namely that of the nanoparticle binding to the sensor surface.

US2003/0215825 addresses a method for detecting or identifying trace quantities of molecular targets. This method is based on the specific affinity of macromolecules for each other such as Watson-Crick binding between complementary nucleic acids and antigen-antibody binding. According to this method, a pair of probes showing high affinity and specificity for a specific molecular target is synthesized. One of the pair is bound to the surface of a sensing device and the other is attached to particles which may be magnetic labels. In the method the molecular target is sandwiched between the functionalised label and the functionalised solid surface.

This method does not solve the above-identified drawbacks. An additional disadvantage is the need to provide probes that specifically bind the target with high affinity and the need to modify the sensor surface by binding one of them to it. This is a complicated process that has to be carried out for each new target that is to be determined.

It is an object of the invention to overcome at least one of these drawbacks. It is a further object to provide a sensor device which is suitable for use in a multiplicity of assays and which is not just suitable for the determination of one specific target composition.

A further object is to establish a molecular sandwich format between a label and a sensor surface, with the special problem that the target capture rate as well as the process of sandwich formation on the sensor surface should be fast and preferably happen in solution.

Another objective is to provide a competitive assay format in which the target and target homologue capture rate as well as the label binding to the sensor surface be rapid.

### SUMMARY OF THE INVENTION

We have surprisingly found that at least one of these objectives is met by a magnetic sensor device which has a surface which is functionalised with one moiety of a strong binding couple, which moiety preferably shows little or no cross reactivity with the target or target homologue molecule.

Therefore in a first aspect the invention relates to a magnetic sensor device for detecting a target in a sample suspected of comprising the target, comprising a sensor surface that is functionalised with at least one moiety (A) of a strong binding couple, which moiety (A) preferably shows little or no affinity for the target molecule and for a target homologue.

In a further aspect the invention relates to a method for detecting a target in a sample suspected of containing the target, using the claimed device.

In another aspect the invention relates to a kit of parts suitable for detecting a target in sample suspected of containing the target.

### DESCRIPTION OF THE FIGURES

FIG. 1 shows a sandwich assay.

FIG. 2 shows a catch assay.

FIG. 3 shows a sandwich assay using a strong binding couple 6, 7.

FIG. 4 shows an assay as in FIG. 3 where a particle with multiple moieties (6) is applied.

FIG. 5 shows an embodiment where 2 surfaces are present, one for binding nonbound moiety (6) and one for binding label (4).

FIG. 6 shows the use of the claimed assay method for detection of different targets.

FIGS. 7 and 8 show various embodiments of competitive assays.

FIG. 9 shows the use of a strong binding couple in a competitive assay. In the upper row the target concentration is high, in the lower row it is low.

FIG. 10 shows an embodiment of a competitive assay where target homologue is directly bound to moiety (B) of the strong binding couple.

FIGS. 11/12 show a competitive assay where a particle with multiple moieties (6) is applied.

FIGS. 13/14 show embodiments of a competitive assay where two surfaces are present, one for binding nonbound moiety (6) and one for binding label (4).

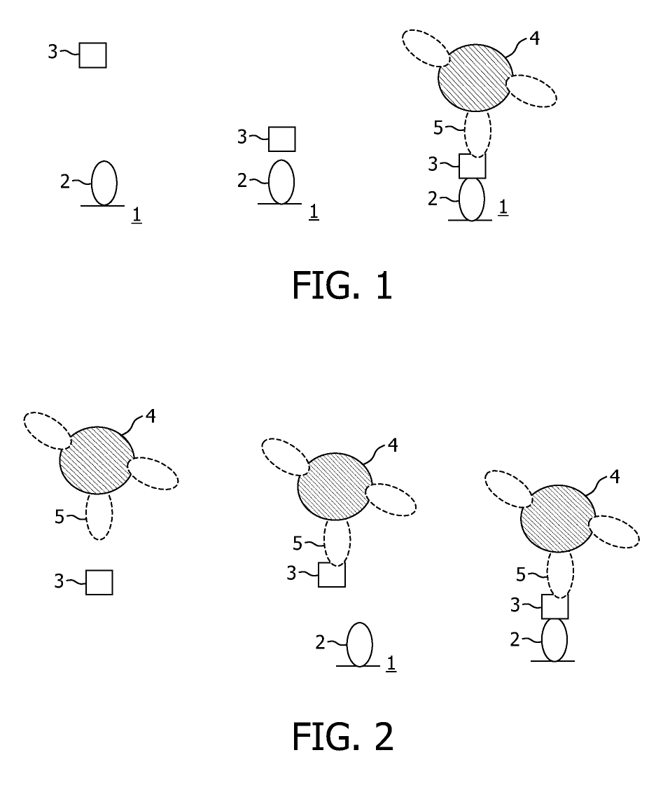

### DETAILED DESCRIPTION

“Target molecule” may be any molecule of which concentration or presence as such is to be determined. Examples of target molecules are molecular targets such as proteins, enzymes, hormones, peptides, nucleic acids and cellular targets such as pathogen cells, bacterial cells and fungal cells. The target molecule may exist as such in a sample that is analysed or may be formed in situ in a sensor device e.g. via a reaction that takes place in the device. If the sensor is used to monitor a reaction, the target may for example be the starting product of the reaction or a reaction product.

Where reference is made to “in solution” what is meant is that the reaction or assay is carried out in a liquid environment. The reagents that take part need not be dissolved in the fluid medium but may also be present in a suspended or dispersed state.

A strong binding couple is a combination of two moieties (molecules) A and B with specific binding between the two moieties wherein moiety A binds to moiety B more strongly or preferentially than to other molecules and shows little or no cross reactivity with other molecules. In general the affinity constant (Ka) for specific binding between moiety A and B is at least 10⁶ l/mol, more preferred at least 10¹⁰ l/mol, even more preferred at least 10¹¹ l/mol, even more preferred at least 10¹² l/mol, even more preferred from 10¹³ to 10¹⁷ l/mol.

The moiety (A) and (B) which are part of a strong binding couple (BC) show little or no affinity for the target molecule. In the context of the invention, “little or no affinity” is defined as having an affinity constant (Ka) of less than 10³ L/mol.

Complementary binding couple moiety refers to a composition comprising moiety (B) and either of

i) a binding probe capable of binding to a part of the target or to a part of a target homologue or

ii) a target homologue. In some types of assays the binding probe of the complementary binding moiety will bind to the target itself. This is for example the case for a standard sandwich assay wherein the amount of target is directly determined.

In competitive assays, instead of the amount of target, the amount of a target homologue is determined. For these types of assays the binding probe of the complementary binding couple moiety preferably binds to both target and target homologue. It will be appreciated that this binding preferably does not take place simultaneously in one molecule.

Target homologue is defined as either a construct which contains at least a part of the target, preferably the part that distinguishes it from other related molecules or a construct that the binding probe binds similarly strong to as the target. Similarly strong binding is defined as having a Ka preferably a factor of 10³, more preferred a factor of 10², most preferred a factor of 10 or smaller in difference. Without wishing to be bound by any theory it is believed that very often a target and target homologue share the same epitopes for binding to a probe.

Label bound probe refers to a composition comprising a detectable label and a binding probe capable of binding to a part of the target or target homologue.

In a first aspect the invention relates to a magnetic sensor device. The sensor device comprises a sensor surface that is functionalised with at least one moiety (A), which is part of a strong binding couple. The binding couple is formed by moiety (A) and (B). It is preferred for the invention that moiety (A) and (B) show little affinity for the target molecule or target homologue.

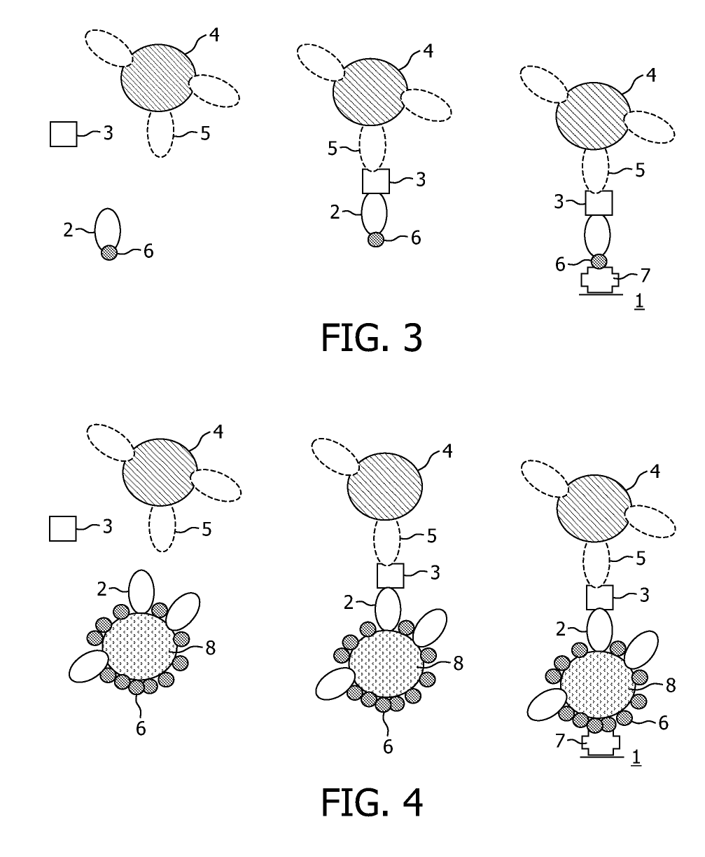

One embodiment of the invention is illustrated in FIG. 3. In this embodiment, the target (3) is captured onto detectable label (4) via a first binding probe (5). The label with probe attached thereto is preferably suspended in solution. While in solution, a complex is added comprising a moiety (B) (item 6) and a binding probe (2) capable of binding to a second part of the target. (sketched as 2 coupled to 6) to complete the sandwich format. This complex is also referred to as complementary binding couple moiety. After binding between label and moiety (B), the label-target-complementary binding couple moiety complex that results, binds to the sensor surface 1 via a strong binding couple, consisting of moieties 6 and 7.

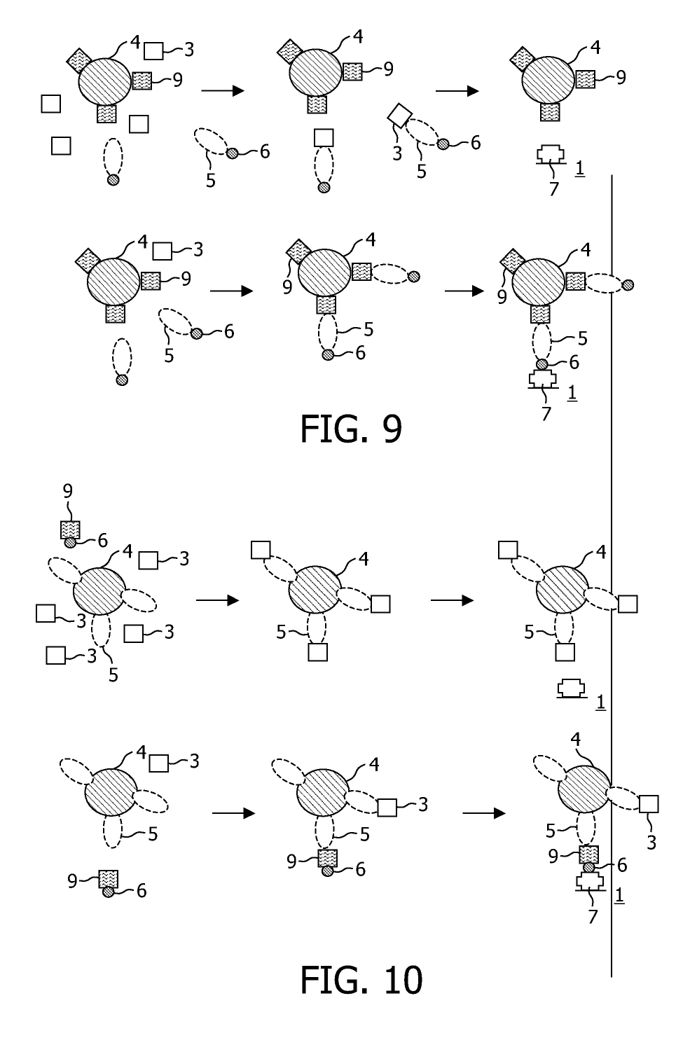

Another embodiment of the invention relates to the competitive assay. This is described in FIG. 9. The top row of FIG. 9 shows an embodiment with high target concentration, the lower row shows an embodiment with low target concentration. In this embodiment, the target (3) and target homologue (9), which is attached to a detectable label 4, compete for capture by a complex containing binding probe 5 and moiety (B) (item 6). After binding of 5,6 to target homologue, the resulting labels containing target—homologue—complementary binding couple, bind to the sensor surface 1 via a strong binding couple, consisting of moieties 6 and 7.

A further embodiment pertaining to the competitive assay is shown in FIG. 10 for cases with high concentrations (top row) and low concentrations (bottom row) of targets. In this embodiment target homologue 9 is complexed with moiety B (item 6) and this complex competes with the target (3) for binding to complementary binding couple (5) which is attached to a detectable label. After target or target homologue binding to the detectable label, the label binds to the sensor surface (1) through the strong binding couple, moieties (6) and (7).

The advantage of this solution is (i) that target's and target homologue's binding rates are high due to their capture onto or on the surface of a detectable label in solution, and (ii) that the binding of a label onto the sensor surface is rapid and efficient due to the use of a strong binding couple.

In the strong binding couple, any chemical species can be chosen to be A and any can be B as A is defined by the existence of B. It is however preferred that in choosing which species is A and which is B, B is chosen such that in coupling it to the complementary binding probe/or target homologue the functionality of the complementary binding probe/or target homologue is not significantly reduced or changed. For example if the strong binding couple is a hapten-antibody, it is preferable to render A the antibody and B the hapten as haptens are generally small and can easily coupled to other biological molecules. Examples of preferred strong binding couples are avidin/biotin, hapten/antibody, protein or peptide/antibody, protein/carbohydrate, protein/protein, nucleic acid/nucleic acid, protein/nucleic acids and hapten/nucleic acids.

The interaction between the protein avidin and the molecule biotin is widely applied to link biological molecules to other moieties. The affinity constant (Kₐ) of avidin with biotin is one of the highest known at approximately 10¹⁵ L/mol and thus the binding is considered irreversible under normal assay conditions. In addition to the high affinity, there are four binding sites available for biotin on each avidin molecule. The biotinylation or chemical labelling of proteins with biotin is facile and does not reduce the biological activity. Avidin can also be chemically coupled to other proteins through standard linking agents involving carbodiimide. There are a number of varieties of avidin commercially available including streptavidin and neutravidin, which differ in degree of glycosylation, isoelectric point and non-specific binding characteristics. Another alternative is the strep-tag II/strep-tactin couple.

High affinity antibodies can be raised to haptens or small molecules including, dyes, drugs, hormones and vitamins. In general, antibodies to nearly any hapten can be created and a number of high affinity antibodies exist with Kₐ greater than 10¹¹ L/mol for molecules such as digoxigenin, 2,4-dinitrophenyl (DNP) and fluorescein-5-isothiocyanate (FITC). There are several known simple haptenylation procedures for chemically labelling molecules with haptens. Attachment of the antibodies can be accomplished also with chemical techniques similar to that described for avidin or with recombinant techniques for coupling to proteins.

The specificity and high binding affinity of proteins or peptides to their antibodies is the basis for many immunoassays. The affinity constant of such interactions can be as high as 10¹³ L/mol and can vary many orders of magnitude depending on the particular peptide or protein used.

Protein-protein binding occurs between specific types of proteins. For example Protein A and protein G are known for their high affinity to the Fc portion of immunoglobulins. Similarly concanavalin A is a lectin which binds to the carbohydrate fraction of glycoproteins although not as strongly as protein A and G binds to immunoglobulins. These interactions are less specific and could be used only in assays where the sample does not contain an Fc region or where the sample is not a glycoprotein. In order to achieve the desired specificity and reduce the cross reactivity, antibodies and proteins within the assay for which these binding interactions are not intended may be modified. For example, one can synthesize recombinant antibodies in which the Fc or glycosylated regions are removed.

The binding of nucleic acid strands, DNA, RNA, or PNA, to strands with a complementary sequence is based on Watson-Crick base pairing. The strength of binding of a strand to its complement is determined by the number of bases in the strand, its specific base content and the type of nucleic acid with PNA binding stronger than RNA which is in turn stronger than DNA binding. It is also possible for strand of different types of nucleic acids to hybridise if they are complementary.

Nucleic acids that fold into a secondary structure have the ability to bind to protein and hapten targets with affinities similar to that of antibodies. These nucleic acids, referred to as aptamers are generally short single stranded RNA and DNA units that can be synthesized using standard techniques. The base sequence of the aptamer for a specific target is selected from a library of strands using an evolutionary selection technique (SELEX) based on their binding affinity to the target.

The sensor device preferably comprises means for introducing a sample.

Optionally the device may be adapted such that parallel measurement of multiple targets can be performed. In this embodiment, the sensor surface is functionalised with at least 2 different moieties (A) and (A′) that belong to different binding couples. In this embodiment, specific binding couples are selected for each different target that is analysed. Optionally the device comprises a multitude of sensors, preferably at least one for each target, whereby on the surface of each sensor that is part of the device and which corresponds to a specific target, is deposited a different binding moiety (A), (A′), (A″) etc. In use, each binding moiety (A), (A′) etc would be complemented with a corresponding binding moiety (B), (B′) etc which is part of the complementary binding couple moiety for a specific target.

This multiple target parallel analysis is illustrated for a sandwich assay in FIG. 6.

In this assay multiple targets (3) become sandwiched between binding probes (2) and (5), wherein probe (5) is coupled to nanoparticle label (4). Probe (2) and (5) are freely present in solution as opposed to being linked to a surface. In a preferred embodiment probe (2) is coupled to nanoparticle (8) containing the moiety (6) (moiety (B) of the strong binding couple) at high density.

In this embodiment, moiety (6) binds to moiety (7) (moiety (A) of the strong binding couple, with moiety (7) coupled to the sensor surface (1). Moieties 2, 5, 6, and 7 are distinct for each target 3. A similar scheme exists for competitive assay in which multiple strong binding couples are used to attach labels with various distinct target homologue-complementary binding probe complexes to a sensor surface.

According to another embodiment, the device comprises a single sensor. In use a variety of first binding probes are each attached to a label with different properties thereby enabling detection of different targets in a sample.

It will be appreciated that it is less desired and potentially disturbing for a measurement, if a binding moiety (A) and (B) encounter and efficiently bind before a sandwich is formed of the label-bound probe, the target, and the complementary binding couple moiety, or before the complementary binding probe has captured the target or target homologue in a competitive assay. This preliminary binding may block part of the sensor surface. This situation especially arises when there is a relatively high concentration of binding moiety (B) present. To avoid this, it is preferred that the sensor device comprises a compartment for sandwich formation or target/target homologue capture and a further compartment for binding of a complementary moiety (B) to the moiety (A) of the strong binding couple.

The moiety (A) of the strong binding couple may be linked to the sensor surface in any suitable way. This attaching (also referred to as linking, coating or bonding) may be by any suitable method such as covalent linking or non-covalent linking. It is preferred that the attaching is via directed interaction rather than by random binding. An example of a link is via a sulfur bridge or bond when a cysteine residue is present at the terminus of the moiety A.

The sensor device may contain any suitable detector for detecting a label. Suitable detectors are magnetic detectors, optical detectors, radioactiveness detectors, or electrical detectors. In the current invention it is highly preferred that the detector is a magnetic detector.

In a further aspect the invention relates to a method for detecting a target in a sample suspected of containing the target comprising

a) Attaching to a detectable label a first binding probe capable of binding to a first part of the target or to a first part of a target homologue to form a label-bound probe;

b) Attaching to a support at least one moiety (A) of a strong binding couple (BC), which moiety (A) shows little or no affinity for the target molecule.

c) Providing a complex comprising at least one moiety (B) which is the binding partner of moiety (A) in the strong binding couple (BC), and either of

i) a binding probe capable of binding to a part of the target or to a part of a target homologue or

ii) a target homologue, to form a complementary binding couple moiety;

d) Bringing into contact the label-bound probe, the sample suspected of containing target, the support and the complementary binding couple moiety;

e) Detecting label that is bound to the support, wherein the label is a magnetic label, or is linked to a magnetic particle.

In a preferred aspect the invention relates to a method for detecting a target in a sample suspected of containing the target comprising

a) Attaching to a detectable label a first binding probe capable of binding to a first part of the target or to a first part of a target homologue to form a label-bound probe;

b) Attaching to a support at least one moiety (A) of a strong binding couple (BC), which moiety (A) shows little or no affinity for the target molecule.

c) Providing a complex comprising at least one moiety (B) which is the binding partner of moiety (A) in the strong binding couple (BC), and at least one second binding probe capable of binding to a second part of the target or to a second part of a target homologue to form a complementary binding couple moiety;

d) Bringing into contact the label-bound probe, the sample suspected of containing target, the support and the complementary binding couple moiety;

e) Detecting label that is bound to the support.

This method is also referred to as “sandwich” method.

Another aspect of the invention relates to a method for detecting a target in a sample suspected of containing the target comprising

a) Attaching to a detectable label at least one target homologue

b) Attaching to a support at least one moiety (A) of a strong binding couple (BC), which moiety (A) shows little or no affinity for the target or target homologue molecule.

c) Providing a complex comprising at least one moiety (B) which is the binding partner of moiety (A) in the strong binding couple (BC), and at least one binding probe capable of binding to the target and to target homologue, but not simultaneously,

d) Bringing into contact the label-bound target homologue, the sample suspected of containing target, the support and the complementary binding probe;

e) Detecting label that is bound to the support

A further part of the invention is an additional “competitive” method for detecting a target in a sample suspected of containing the target comprising

a) Attaching to a detectable label a binding probe capable of binding to the target and the target homologue, but not simultaneously, to form a label-bound probe;

b) Attaching to a support at least one moiety (A) of a strong binding couple (BC), which moiety (A) shows little or no affinity for the target molecule or target homologue.

c) Providing a complex comprising at least one moiety (B) which is the binding partner of moiety (A) in the strong binding couple (BC), and at least one target homologue

d) Bringing into contact the label-bound probe, the sample suspected of containing target, the support and the complex with the target homologue and strong binding partner moiety.

e) Detecting label that is bound to the support.

The two previous methods are also referred to as “competitive” methods.

In view of the above-described risk of preliminary binding between moiety (A) and (B) it is preferred that the step (d) comprises two separate steps comprising (d1) bringing into contact

i) for the sandwich method the label-bound probe, the sample and the complementary binding couple moiety to allow the formation of a label-target second binding probe complex,

ii) for the first competitive method as described above, the label bound target homologue, the sample and the complex comprising the binding probe and the binding moiety B

iii) for the second competitive method as described above, the label-bound probe, the sample and the complex comprising the target homologue and binding moiety B, (d2) bringing the resulting complex containing the label in contact with the support that has attached thereto at least moiety (A).

Preferably the sample and all other components that take part in the analysis are present in the device in a liquid form during the analysis. However, it is possible that some of the components that take part in the analysis are initially present in a dry form.

To avoid a high background signal during detection it is advantageous to remove excess label and binding moiety B that have not bound to target or target homologue. Therefore in a preferred embodiment, excess complementary binding couple moiety and excess label-bound probe that in the sandwich method are not bound to target or in the competitive methods are not bound to target homologue are removed before step (d2). Below it is described in a preferred embodiment how this may be achieved in a magnetic sensor.

In another aspect the invention relates to a method for detecting a target in a sample suspected of containing the target comprising introducing the sample in a sensor device according to the invention, and further introducing into the device

a) a detectable label having attached thereto a first binding probe capable of binding to a first part of the target or to a first part of a target homologue,

b) a complex comprising a moiety (B) which is the binding partner of moiety (A) in the strong binding couple (BC), and either of

i) a binding probe capable of binding to a second part of the target or to a second part of a target homologue or

ii) a target homologue.

The detection of target or target homologue is based on the detection of presence or absence of a detectable label. The detectable label may be any label such as fluorescent label, a calorimetric label, chemiluminescence label, enzymatic label with the corresponding converted products (e.g. chemiluminescent, fluorescent, electrostatically charged species, and electron donating/accepting species) a magnetic label, a radioactive label, electrostatically charged label, donating/accepting label. Most preferred, the label is a magnetic label or a label linked to a magnetic particle.

In the case of magnetic sensors the analysis is based on the detection of a label that is bound to a sensor surface. The current assay set up involves linkage to a surface and therefore the use of a magnetic label is highly preferred.

If the label is a magnetic label, use is generally made of magnetic particles which may have a size between 10 nm and a few micrometers, more preferred between 30 nm and 300 nm. In a preferred method the magnetic particle is larger than the individual biological molecules involved in the assay.

Magnetic particles may be actuated by magnetic fields. When forces are applied in such a way that the magnetic particles are brought to the sensor surface, the biological binding rate can be enhanced. Also, magnetic forces can be applied to distinguish between weak and strong binding, so-called magnetic stringency.

The magnetic labels may be any shape or form. The labels include any form of one or more magnetic particles e.g. magnetic, diagmagnetic, paramagnetic, superparamagnetic, ferromagnetic, ferromagnetic that is any form of magnetism which generates a magnetic dipole in an electric field, either permanently or temporarily.

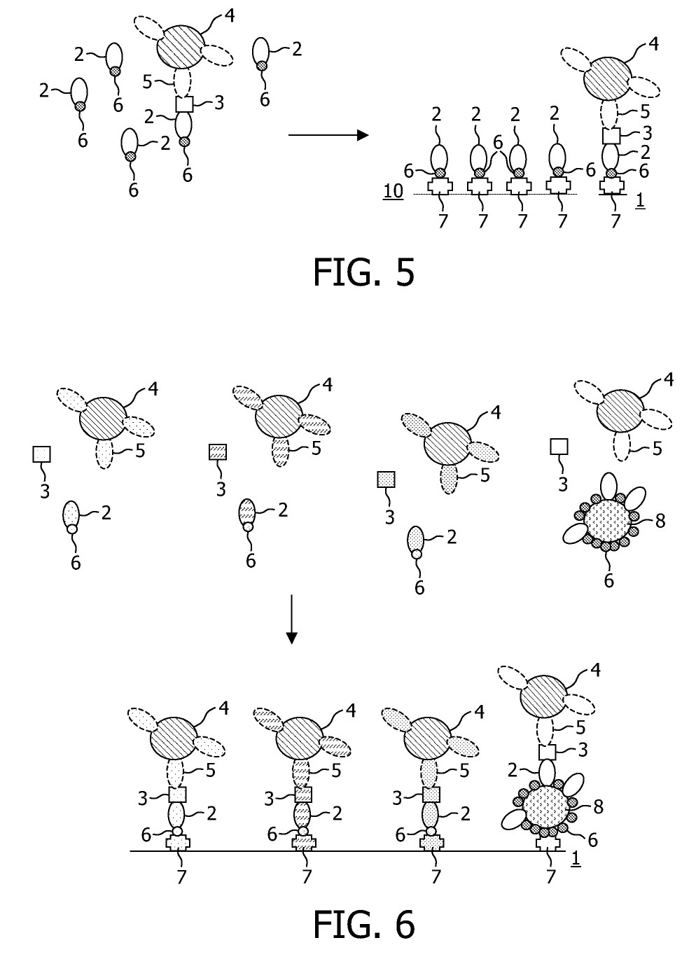

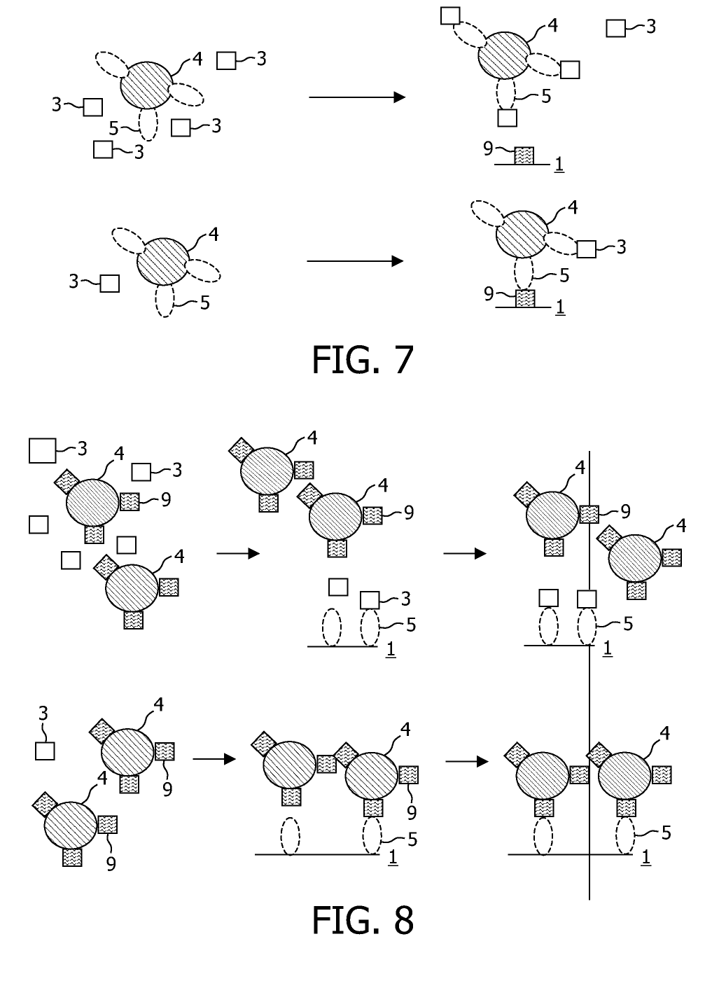

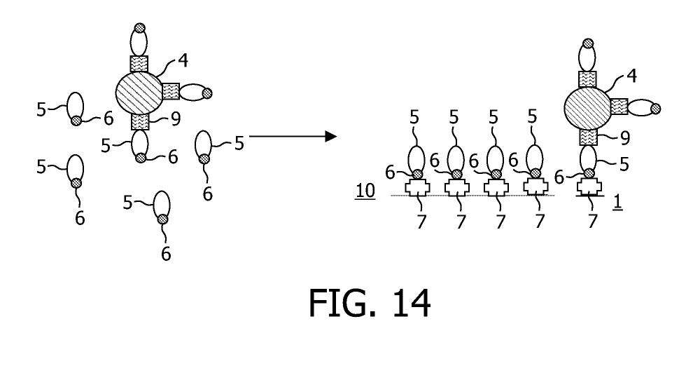

For reasons specified above it is preferred that the method includes a step where complementary binding complex containing moiety (B) that is not bound to target or target homologue, or target homologue with moiety B that is not attached to a label is removed. Where the label is a magnetic label a washing out of moiety (B) that is not bound to target or target homologue, or target homologue with moiety B that is not attached to a label is easily achieved in the method that is illustrated in FIGS. 5, 13, and 14, the sandwich and competitive assay formats respectively. In this embodiment, the sensor comprises a second surface (10) having attached thereto a binding moiety (A) (7) upstream of the biosensor surface (1) that also has attached thereto binding moiety (A). The surface (10) is magnetically actuated to repel the magnetic label (4) thus allowing only free, unbound moiety (B) (6) to bind there.

Preferably at the same time magnetic surface 1 is magnetically actuated such that the magnetic label (4) is attracted through the medium towards biosensor surface 1. This combination of actuation of different sensor surfaces facilitates binding of the complex comprising magnetic label and removal of unbound complex binding couple moiety comprising moiety (B).

The label and the first binding probe or target homologue may be attached to each other with or without a linker. Optionally the attachment is done via a core molecule such as a nanoparticle to which at least one label and at least one first binding probe or at least one target homologue are attached. In an alternative embodiment, the label and the binding probe or target homologue are linked via a linker.

In an example the label is a magnetic label and it is conjugated with a binding probe. To form a conjugate of a magnetic label and a binding probe, the surface of the magnetic label may be modified. This modification can be done, for example, through covering the surface of the magnetic label with dextrane, alkanethiols with suitable end groups, certain peptides etc. A dextrane molecule may covalently bind to a binding probe such as the antibody through cyanobromide activation or carboxylic acid activation.

The first and second binding probe are probes that preferably bind to different parts (epitopes) of a target or target homologue molecule. Examples of suitable binding probes are Affibodies™, antibodies, receptor molecules, aptamers and chelators.

In the case where the target is a nucleic acid, the first and second binding probe would comprise nucleic acids having a base sequence that is complementary to a part of the sequence of the target. In some embodiments in competitive assays it is desired that the binding probe may have affinity to both the target and the target homologue (but as said earlier, preferably not at the same time in one molecule).

Preferably the binding probes are antibodies specifically binding the target.

The second or complementary part of the strong binding couple is binding moiety (B). In the method of the invention a complex is provided comprising a moiety (B) which is the binding partner of moiety (A) in the strong binding couple (BC), and either of

i) a binding probe capable of binding to a part of the target or to a part of a target homologue or

ii) a target homologue, to form a complementary binding couple moiety. The complex may contain a conjugate of moiety B and the second binding probe or target homologue. In the context of the invention a conjugate is a compound wherein there is a link, preferably a covalent link between the moiety B and the second binding probe.

To increase the probability that moiety (A) and (B) encounter and efficiently bind the label to the sensor surface, a high concentration of (A) and (B) is desirable. In a preferred embodiment, an increase in density of strong binding couples is accomplished by a complex, which comprises a core having attached thereto a multitude of binding moieties (B).

In a preferred embodiment the complex comprises a core having attached thereto at least 2 binding moieties (B) and at least 1 of either of

i) a binding probe capable of binding to a part of the target or to a part of a target homologue or

ii) a target homologue.

In an even more preferred embodiment at least 3, more preferred at least 4, even more preferred at least 5, even more preferred at least 6 binding moieties (B) are attached to the core.

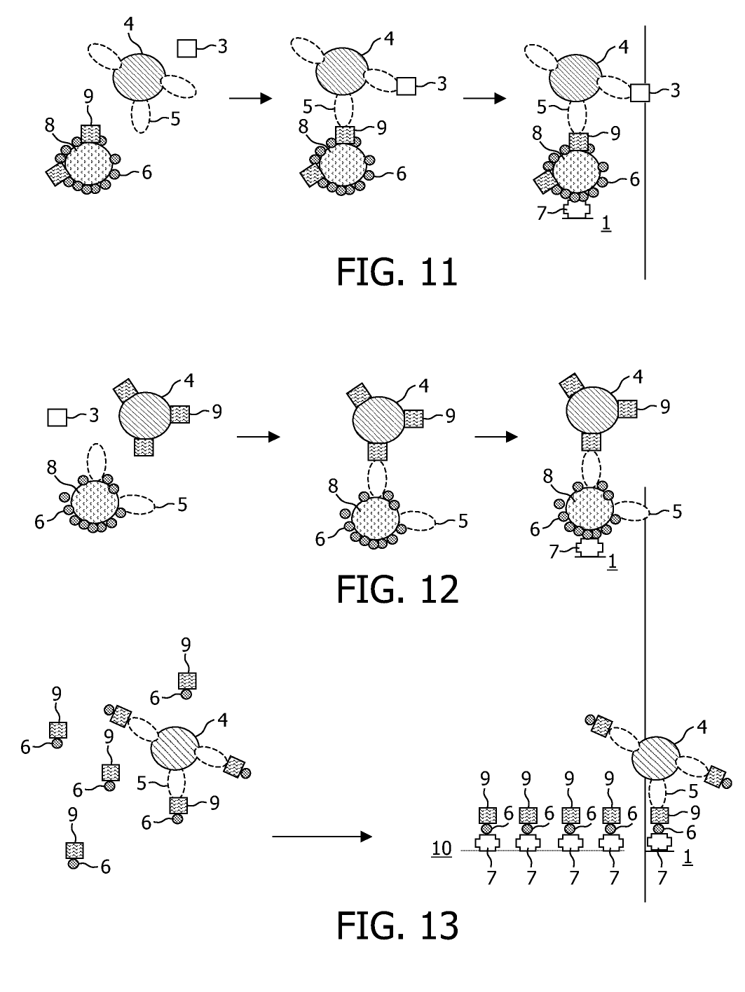

An example of a method where binding moiety (B) is present in high amount on the surface of a core structure is presented in FIG. 4. In this assay the target 3 becomes sandwiched between binding probes 2 and 5, wherein moiety 5 is coupled to nanoparticle label 4 and moiety 2 is coupled to nanoparticle 8 containing the moiety (B) 6 at high density. Thereafter, moiety 6 binds to moiety 7, with moiety 7 coupled to the sensor surface 1. FIGS. 11 and 12 depict similar schemes for the competitive format.

In order to reduce steric hindrance during the binding process of moiety (A) and (B) it is preferred that the core of the complex has a small diameter in the order of from 2 to 200 nm.

The core can be made of any suitable material. Example of suitable core material include polystyrene, silica and magnetic particles.

In a preferred embodiment both the core of the complex and the label have magnetic properties. This facilitates strong binding between (A) and (B) and further facilitates removal of moiety (B) that is not bound to target, or for the competition assay which is not bound to label.

In one embodiment, the moiety (B) is attached to a core with magnetic properties. In that embodiment, magnetic fields can be used to increase the rate of binding between moiety (A) and (B). It is then preferred to select nanoparticles as core material that have a different frequency dependence of magnetic properties than labels. Preferably, the nanoparticles respond only to low-frequency fields, while the labels respond to low as well as to high frequencies. Then the sandwich formation can occur under particle attraction by low-frequency fields, away from the sensor surface. Binding to the surface can occur under attraction by high-frequency fields. As a result, non-sandwich nanoparticles or nanoparticles not attached to labels will not bind to the sensor surface and can easily be removed.

In another aspect the invention relates to a kit of parts suitable for detecting a target in a sample suspected of containing the target, comprising

a) a device comprising a surface that is functionalised with at least one moiety (A) of a strong binding couple

b) reagents comprising strong binding couple moiety (B).

One of the main advantages of such a device is that it allows for the separation of a binding and detection step. The binding step may take place in solution, which can be more effective due to the higher mobilities of the binding components and the larger surface area for binding and the detection may take place at a surface in the device. The physical separation of these two events in one device reduces the total time that is needed for the assay that is carried out in the device (including the detection step).

The reagents comprising strong binding couple moiety (B) are specified above in more detail.

In a further preferred aspect the invention relates to a kit of parts suitable for detecting a target in a sample suspected of containing the target, comprising

a) A sensor device according to the invention

b) A compartment comprising a complex comprising a moiety (B) which is the binding partner of moiety (A) in the strong binding couple (BC), and either of

i) a binding probe capable of binding to a second part of the target or to a second part of a target homologue or

ii) a target homologue.

Alternatively the complex of moiety B and a second binding probe or a target homologue is not present in a separate compartment but included in the device in a way where binding to target, target homologue or label is masked. Such a device allows the initial reaction or process to take place, followed by unmasking of the complex to make binding to target, target homologue or label possible. This masking can be done in a variety of ways, e.g. by encapsulation or physical means that prevent binding.

Such a kit is preferably sold with instructions on the type of label that is suitable and further instructions that in an analysis, a label linked to a first binding probe that is capable of binding a first part of a target, should also be used.

Optionally the kit additionally comprises a detectable label compound and preferably instructions on how this label may be linked to a suitable binding probe. In a preferred embodiment the detectable label is functionalised to facilitate attachment of a suitable binding probe thereto.

In a further preferred embodiment the kit of parts additionally comprises a compartment comprising a detectable label attached to a first binding probe capable of binding to a first part of a target to form a label-bound probe.

The invention is illustrated by the following non limiting example.

### EXAMPLE 1

### Detection of Human Parathyroid Hormone (PTH) Using the Strong Binding Couple Neutravidin/Biotin

1. Materials
Neutravidin
Biotinylated-BSA (bovine serum albumin)
Human PTH
300 nm magnetic particles coated with α-PTH capture antibody
PTH Biotinylated α-PTH tracer antibody

2. Method

A. Coating of the GMR Surface with Neutravidin
GMR Au surfaces were coated with BSA-Biotin (1 mg/mL; 1 hr) and blocked with 3% BSA in PBS (phosphate buffered saline)
Surfaces were washed with 0.05% Tween20 in PBS
Surfaces were then incubated for 30 min at room temperature with neutravidin at 100 g/mL and then washed

B. Binding of Magnetic Particles Coated with Capture Ab to PTH and Biotinylated Tracer Ab
Magnetic particles were incubated with PTH for 30 min at room temperature and 300 rpm in assay buffer. Thereafter, biotinylated α-PTH tracer Ab was added and the mixture was incubated for a further 60 minutes.
The suspension was washed 3 times and resuspended in assay buffer.

C. Binding of Magnetic Particle Labelled PTH to Neutravidin-GMR Surfaces
A GMR sensor with neutravidin on the surface was exposed to 100 L of reacted assay solution from B.
Using external electromagnetic coils, magnetic particle labels were attracted to the surface of the sensor for a total of 15 minutes using a field of approximately 8e5 A/m, after which, a magnetic force of the same magnitude was applied in the opposite direction with a coil above the sensor. This magnetic wash was applied for 10 minutes.
The number of bound magnetic labels was detected with the GMR sensor

The GMR signal from magnetic labels attached to the GMR-neutravidin surface as a function of PTH analyte concentration was determined. The values were as follows:

| | |
|---|---|
| PTH (pM) | Average GMR signal % |

| | |
|---|---|
| 0 | 1.55 |
| 4 | 2.30 |
| 20 | 4.76 |
| 100 | 16.85 |
| 1000 | 28.34 |

1. Magnetic sensor device for detecting a target in a sample suspected of comprising the target, comprising a sensor surface that is functionalised with at least one moiety (A) of a strong binding couple, which moiety (A) shows little or no affinity for the target molecule.

2. Sensor device according to claim 1 wherein the strong binding couple is selected from the group comprising avidin/biotin, hapten/antibody, protein or peptide/antibody, protein/carbohydrate, protein/protein, nucleic acid/nucleic acid, hapten/nucleic acid and protein/nucleic acid.

3. Sensor device according to claim 1, which further comprises means for introducing the sample.

4. Sensor device according to claim 1 wherein the surface is functionalised with at least 2 different moieties (A) and (A′) that belong to different binding couples.

5. Sensor device according to claim 1 which is suitable for use in a sandwich assay, the device comprising a compartment for sandwich formation and a further compartment for binding of a complementary moiety (B) to the moiety (A) of the strong binding couple.

6. Sensor device according to claim 1 which comprises a masked complex comprising a
i) moiety (B) which is the binding partner of moiety (A) in the strong binding couple (BC),
ii) and at least one second binding probe capable of binding to a second part of the target or of a target homologue;
which masking prevents binding of the complex to a target or a target homologue.

7. Method for detecting a target in a sample suspected of containing the target comprising
a) Attaching to a detectable label (4) a first binding probe (5) capable of binding to a first part of the target (3) or to a first part of a target homologue (9) to form a label-bound probe;
b) Attaching to a support at least one moiety (A) of a strong binding couple (BC), which moiety (A) preferably shows little or no affinity for the target molecule.
c) Providing a complex comprising at least one moiety (B) which is the binding partner of moiety (A) in the strong binding couple (BC), and either of
i) a binding probe capable of binding to a second part of the target or to a second part of a target homologue or ii) a target homologue, to form a complementary binding couple moiety;
d) Bringing into contact the label-bound probe, the sample suspected of containing target, the support and the complementary binding couple moiety;
e) Detecting label that is bound to the support, wherein the label is a magnetic label, or is linked to a magnetic particle.

8. Method according to claim 7 wherein step (d) comprises two separate steps comprising
a) (d1) bringing into contact the label bound probe, the sample and the complementary binding couple moiety to allow the formation of a label-target or target homologue-second binding probe complex,
b) (d2) bringing the complex that is formed in contact with the support that has attached thereto at least moiety (A).

9. Method according to claim 8, which further comprises a step wherein excess complementary binding couple moiety and excess label-bound probe that are not bound to target or target homologue, are removed before step (d2).

10. Method according to claim 7 wherein in (c) a complex is provided which comprises a core having attached thereto
i) at least 2 binding moieties (B) and
ii) at least one binding probe capable of binding to a second part of the target or to a second part of a target homologue.

11. Method according to claim 7 wherein the complex comprises a core having attached thereto at least 2 binding probes capable of binding to a second part of the target or to a second part of a target homologue.

12. A method for detecting a target in a sample suspected of containing the target comprising introducing the sample in a sensor device according to claim 1, and further introducing into the device
a) a detectable label having attached thereto a first binding probe capable of binding to a first part of the target,
b) a complex comprising a moiety (B) which is the binding partner of moiety (A) in the strong binding couple (BC), and either of
i) a binding probe capable of binding to a second part of the target or to a second part of a target homologue or ii) a target homologue.

13. Kit of parts suitable for detecting a target in a sample suspected of containing the target, comprising
a) a device comprising a surface that is functionalised with at least one moiety (A) of a strong binding couple
b) reagents comprising strong binding couple moiety (B).

14. Kit of parts suitable for detecting a target in a sample suspected of containing the target, comprising
a) A sensor device according to claim 1
b) A compartment comprising a complex comprising a moiety (B) which is the binding partner of moiety (A) in the strong binding couple (BC), and either of
i) a binding probe capable of binding to a second part of the target or to a second part of a target homologue or
ii) a target homologue.

15. Kit of parts according to claim 14 further comprising a compartment comprising a detectable label attached to a first binding probe capable of binding to a first part of the target or to a first part of a target homologue to form a label-bound probe.
